(19) United States
(12) Patent Application Publication
(10) Pub. No.: US 2020/0367478 A1
(43) Pub. Date: Nov. 26, 2020

(54) METHOD FOR ESTABLISHING ULCERATIVE COLITIS ANIMAL MODEL AND USE OF SAID MODEL

(71) Applicant: Dali University, Dali (CN)

(72) Inventors: Yu Zhao, Dali (CN); Funeng Geng, Dali (CN); Zaipin Xu, Dali (CN); Yongmei Shen, Dali (CN); Heng Liu, Dali (CN); Hairong Zhao, Dali (CN); Li Chen, Dali (CN); Chenggui Zhang, Dali (CN); Xiumei Wu, Dali (CN)

(21) Appl. No.: 16/463,578

(22) PCT Filed: Feb. 8, 2018
(86) PCT No.: PCT/CN2018/075705

§ 371 Date: May 23, 2019

(30) Foreign Application Priority Data
Nov. 27, 2017 (CN) 201711205144.0

(51) Int. Cl. A01K 67/027; A61K 49/00

(52) U.S. Cl. CPC A01K 67/027; A01K 2267/03; A01K 2227/10; A61K 49/0008

(57) ABSTRACT

A method is provided for establishing an ulcerative colitis (UC) animal model and use of the model. The ulcerative colitis animal model uses Canis lupus familiaris dogs as the modeling animals and uses acetic acid to induce canine ulcerative colitis. The experimental results can be directly generalized to the human body. The modeling manner is easy to operate, establishes a stable model, is easy to reproduce, and has a low cost, which provides more animal model options for evaluation of responsiveness to drugs and therapeutic effect in research and development of UC therapeutic drugs.

### CROSS-REFERENCE TO RELATED APPLICATIONS

This application is a national phase entry of, and claims priority to, International Application No. PCT/CN2018/075705, filed Feb. 8, 2018, which claims priority to Chinese Patent Application No. 201711205144.0 filed on Nov. 27, 2017. The above-mentioned patent applications are incorporated herein by reference in their entireties.

### TECHNICAL FIELD

The present invention relates to the field of experimental animal models, and more particularly, relates to a method for establishing an ulcerative colitis animal model and associated uses.

### BACKGROUND

Ulcerative Colitis (UC) is a chronic non-specific ulcerative colitis characterized by ulceration and chronic inflammation, which is one type of Inflammatory Bowel Disease (IBD). UC has an unknown etiology, and has main clinical manifestations of diarrhea, abdominal pain, mucopurulent bloody stool and other gastrointestinal symptoms; and the lesion usually involves rectum and sigmoid colon, or spreads throughout the colon, mainly attacks large intestine mucosa and submucosa, and has a stepwise and diffuse distribution. UC has alternately repeated attack period and remission period, and a long course of disease, is difficult to cure, has a high recurrence rate after being cured, has a certain correlation with onset of colon cancer, and has a poor prognosis, and thus is listed as one of modern refractory diseases by the World Health Organization. It has drawn widespread attention from the medical field. Establishment of an animal model applicable to UC research with similar clinical symptoms, good reproducibility and simple operation not only provides a basis for studying disease patterns, but also provides suitable conditions for development and manufacture of new drugs for treating UC.

A commonly-used experimental animal model for UC disease research/efficacy evaluation is established by inducing intestinal ulcer lesions and inflammatory responses in healthy experimental animals by using a chemical stimulation method, an immunization method, a composite method and the like. The method of conducting chemical stimulation with acetic acid destroys the intestinal mucosal barrier structure of an experimental animal, and in turn initiates inflammation and causes inflammatory mediators. The model has lesion pathological changes and inflammatory disorders which are similar to the intestinal inflammatory properties caused by abnormal arachidonic acid metabolism in human, is simply prepared with low cost and high success rate, has a good reproducibility, and can exhibit obvious symptoms in a short period, and the UC induction method of the model is relatively simple.

Currently, the experimental animals used in UC animal models are commonly-used rats and domestic rabbits, and occasionally-used guinea pigs and mice; however, rodents, including lagomorpha and rodentia, cannot be directly promoted to human body since they have physiology, toxicology, and responses to disease totally different from those of human due to their feeding habits which are different from those of human.

Currently, the experimental dogs adopted in an UC animal model established with dogs are adult German Shepherd or Beagle dogs. These dogs are susceptible to external environmental factors during the feeding period. The relatively large body size of these adult dogs causes that the operation procedure of artificial UC induction is difficult to standardize, which in turn makes it difficult to effectively control confounding factors associated with UC. Moreover, the individual immunity of the dogs is relatively poor, which is easy to induce other diseases or even death during a modeling process. Furthermore, in order to avoid generation of other diseases or death in the German Shepherd or Beagle dogs during the modeling process, the chemical agents that induce generation of UC as disclosed in literature methods are relatively lower in dose, and thus the degree of inflammation as caused is greatly different from clinical manifestations of human, such that it is unable to objectively and correctly reflect the disease process or evaluate the therapeutic effect of a drug.

Thus, it would be desirable to provide a method for establishing an ulcerative colitis animal model and use of the model which can better reflect ulcerative colitis in humans.

### SUMMARY

To achieve the above objective, the present invention provides the following technical solutions, in one embodiment. A method for establishing an ulcerative colitis animal model is provided, including the following steps: making a Canis lupus familiaris be subjected to gavage and intestinal cleansing, and be fasted but have access to water for 24 h; and anesthetizing the Canis lupus familiaris, and then making the anesthetized Canis lupus familiaris be subjected to enema with an acetic acid solution, to obtain the ulcerative colitis animal model when the Canis lupus familiaris naturally wakes up.

In one aspect, the Canis lupus familiaris is an adult dog.

In another aspect, the Canis lupus familiaris is 1-2 years old.

Still further, the body weight of the Canis lupus familiaris is 6.0-12.0 Kg.

In a further aspect, the enema manner is injecting the acetic acid solution into the colon of the Canis lupus familiaris, retaining the solution in the colon for more than 10s, and then rinsing the colon with normal saline.

In yet another aspect, the mass concentration of the acetic acid solution is 5-10%, and more preferably, the dose of the acetic acid solution is 1 mL to 2 mL per kilogram of body weight.

In some embodiments, the gavage drug is an MgSO₄ solution.

In one aspect, the MgSO₄ solution has a concentration of 5-10%, and a dose of 5-10 mL/kg.

In another aspect, the anesthetizing the Canis lupus familiaris includes: intramuscularly injecting Shumianning into the Canis lupus familiaris.

The above ulcerative colitis animal model obtained by the present invention in accordance with one embodiment can be used for screening UC therapeutic drugs and evaluating the efficacy of UC therapeutic drugs.

The present invention, in another embodiment, also provides a method for screening and evaluating UC therapeutic drugs, including the following steps: a. setting a to-be-tested drug group, a normal saline control group, and a normal control group, where the to-be-tested drug group and the normal saline control group use the ulcerative colitis animal model established by using the above method, and the normal control group contains a healthy Canis lupus familiaris not subjected to the modeling; observing and recording the physiological basic data and colonic pathology conditions of the to-be-tested drug group, the normal saline control group and the normal control group; b. feeding the to-be-tested drug group with a drug to be tested, and feeding both the normal saline control group and the normal control group with normal saline, and observing and recording the physiological basic data and colonic pathology conditions of the to-be-tested drug group, the normal saline control group and the normal control group after the treatment; and c. analyzing the data and grades of colonic pathological photographs recorded in step a and step b, and evaluating the therapeutic effect of the drug to be tested; where if the physiological basic data of the to-be-tested drug group is closer to the corresponding measurement value of the normal control group than the physiological basic data of the normal saline control group, or the grade of the colonic pathological photograph of the to-be-tested drug group is lower than that of the normal saline control group, then it indicates that the drug to be tested has the effect of treating UC.

The grading criteria of the colonic pathology photographs preferably are : grade 0: the mucosa is pale, the vasoganglion is clear and branched; there is no redness and swollen or congestion under the mucosa, and the surface mucosa is normal; grade 1: the mucosa is still smooth, but is subjected to congestion and redness and swollen in a small area, and has enhanced refraction; grade 2: the mucosa is subjected to congestion and edema, is granular, has increased mucosa fragility, and is easily bleeding upon contact; grade 3: the mucosa is subjected to obvious congestion and edema, is rough, has a few spontaneous bleeding points or suffers from contact bleeding; the mucosa has relatively more inflammatory secretions, is subjected to multiple erosion and small-area ulceration; and grade 4: the mucosa is subjected to congestion and edema in a large area, is rough, suffers from obvious spontaneous bleeding and contact bleeding; and is subjected to multiple punctate erosion and large-area ulceration; where if the grade of the colonic pathology photograph of the to-be-tested drug group is low, it indicates that the drug to be tested has a good effect for treating UC.

The physiological basic data of the animal includes but is not limited to one or more of a body temperature, a body weight, a stool form, an occult blood examination, a blood routine, a blood biochemical index, and a C-reactive protein.

The embodiments of the present invention achieve the following advantages.

Canis lupus familiaris is selected as the model animal. This kind of dog has a wide animal source, mainly includes mongrel dogs, is less affected by external environmental factors and has better individual immunity. Since the digestive system of a dog is similar to that of human, substantially the experimental results of this model can be directly generalized to human body. The modeling manner of establishing a model by inducing canine ulcerative colitis with acetic acid, as provided by the present invention, is easy to operate, establishes a stable model, is easy to reproduce, and has a low cost, which provides more animal model options for evaluation of responsiveness to drugs and therapeutic effect in research and development of UC therapeutic drugs.

### BRIEF DESCRIPTION OF THE DRAWINGS

Various additional features and advantages of the invention will become more apparent to those of ordinary skill in the art upon review of the following detailed description of one or more illustrative embodiments taken in conjunction with the accompanying drawings. The accompanying drawings, which are incorporated in and constitutes a part of this specification, illustrate one or more embodiments of the invention and, together with the general description given above and the detailed description given below, explain the one or more embodiments of the invention.

FIG. 1A is a pictorial view showing a colonoscopy image of an intestinal track taken before modeling with 10 mL of 7% acetic acid.

FIG. 1B is a pictorial view showing a colonoscopy image taken of the intestinal track of FIG. 1A after the modeling.

FIG. 1C is a pictorial view showing a colonoscopy image taken of the intestinal track of FIG. 1B four days after the modeling.

FIG. 1D is a pictorial view showing a colonoscopy image taken of the intestinal track of FIG. 1C seven days after the modeling.

FIG. 2A is a pictorial view showing a colonoscopy image of an intestinal track taken before modeling with 15 mL of 10% acetic acid.

FIG. 2B is a pictorial view showing a colonoscopy image taken of the intestinal track of FIG. 2A after the modeling.

FIG. 2C is a pictorial view showing a colonoscopy image taken of the intestinal track of FIG. 2B four days after the modeling.

FIG. 2D is a pictorial view showing a colonoscopy image taken of the intestinal track of FIG. 2C seven days after the modeling.

FIG. 3A is a pictorial view showing a colonoscopy image of an intestinal track taken before modeling with 20 mL of 5% acetic acid.

FIG. 3B is a pictorial view showing a colonoscopy image taken of the intestinal track of FIG. 3A after the modeling.

FIG. 3C is a pictorial view showing a colonoscopy image taken of the intestinal track of FIG. 3B four days after the modeling.

FIG. 3D is a pictorial view showing a colonoscopy image taken of the intestinal track of FIG. 3C seven days after the modeling.

FIG. 4 shows a series of colonoscopy images corresponding to differing colonoscopy grades (0 through 4) of acetic acid-induced acute ulcerative colitis in Canis lupus familiaris.

### DETAILED DESCRIPTION

The following clearly and completely describes the technical solutions in the embodiments of the present invention with reference to the accompanying drawings in the embodiments of the present invention. To make objectives, features, and advantages of the present invention clearer, the following describes embodiments of the present invention in more detail with reference to accompanying drawings and specific implementations.

The present invention uses Canis lupus familiaris as the model animal, and establishes an ulcerative colitis animal model by inducing canine ulcerative colitis with acetic acid. In particular, the present invention includes the following steps.

A Canis lupus familiaris is subjected to gavage and intestinal cleansing, and is fasted but has access to water for 24 h; and The Canis lupus familiaris is anesthetized, and then the anesthetized Canis lupus familiaris is subjected to enema with an acetic acid solution, to obtain the ulcerative colitis animal model when the Canis lupus familiaris naturally wakes up.

The experimental Canis lupus familiaris used in the present invention is preferably a healthy adult Canis lupus familiaris. In a specific embodiment of the present invention, adopted is a Canis lupus familiaris which is preferably 1-2 years old and has a body weight of preferably 6.0-12.0 Kg.

The present invention has no specific limitation to the specific operations of gavage and enema, and gavage and enema can be operated by a conventional technique known to those skilled in the art. Preferably, the enema manner is injecting an acetic acid solution into the colon of the Canis lupus familiaris, retaining the solution in the colon for more than 10s, and then rinsing the colon with normal saline. The specific enema operation is preferably: after the Canis lupus familiaris is anesthetized, a catheter is inserted into the colon at a depth of 20 cm from the anus of the Canis lupus familiaris, the acetic acid solution is slowly injected into the colon and the acetic acid enema is kept for more than 10s, and then the colon of the Canis lupus familiaris is rinsed with normal saline when the Canis lupus familiaris is positioned at a head low and tail high body position.

In the present invention, a conventional animal anesthesia method in the art can be employed as the anesthesia manner of the Canis lupus familiaris. In a specific embodiment of the present invention, preferably Shumianning is intramuscularly injected into the Canis lupus familiaris. Operation is conducted according to the anesthetic dose stated in the instruction manual.

In the present invention, acetic acid is used to cause ulceration in the intestinal tract of an animal, which in turn causes inflammation. The acetic acid solution used in the present invention has a mass concentration of 5-10%, and more preferably 7-9%. The dose of the acetic acid solution is appropriately adjusted according to the concentration of acetic acid. The dose of the acetic acid solution used in the present invention is preferably 1-2 mL/kg the Canis lupus familiaris, and more preferably 1.3-1.7 mL/kg.

In the present invention, the time for conducting enema with acetic acid is more than 10s, and preferably 20-40 s. The colon is rinsed with normal saline after the enema. The present invention has no specific limitation to the dose of normal saline, and the dose of normal saline is preferably 20-100 mL, and more preferably 50-60 mL.

When the animal wakes up after the modeling, with reference to the sign conditions and after the animal signs and colonoscopy of the animals after the modeling, if symptoms such as hematochezia, intestinal mucosa erosion, and intestinal mucosa hyperemia are found, it is confirmed the modeling is successful.

The above ulcerative colitis animal model obtained by the present invention can be used for screening UC therapeutic drugs and evaluating the efficacy of UC therapeutic drugs.

The present invention also provides a method for screening and evaluating UC therapeutic drugs, including the following steps: a. setting a to-be-tested drug group, a normal saline control group, and a normal control group, where the to-be-tested drug group and the normal saline control group use the ulcerative colitis animal model established by using the method of the present invention, and the normal control group contains a healthy Canis lupus familiaris not subjected to the modeling; observing and recording the physiological basic data and colonic pathology conditions of the to-be-tested drug group, the normal saline control group and the normal control group; b. feeding the to-be-tested drug group with a drug to be tested, and feeding the normal saline control group and the normal control group with normal saline, determining the number of treatment days according to the clinical medication cycle of the drug to be tested, and observing and recording the physiological basic data and colonic pathology conditions of the to-be-tested drug group, the normal saline control group and the normal control group after the treatment; c. analyzing the data and colonic pathological photographs recorded in step a and step b, and evaluating the therapeutic effect of the drug, where if the physiological basic data of the to-be-tested drug group is closer to the corresponding measurement value of the normal control group than the physiological basic data of the normal saline control group, or the grade of the colonic pathological photograph of the to-be-tested drug group is lower than that of the normal saline control group, then it indicates that the drug to be tested has the effect of treating UC.

The colonic pathology photographs formed by the ulcerative colitis animal model used in the present invention are preferably graded according to the following grading criteria: grade 0: the mucosa is pale, the vasoganglion is clear and branched; there is no redness and swollen or congestion under the mucosa, and the surface mucosa is normal; grade 1: the mucosa is still smooth, but is subjected to congestion and redness and swollen in a small area, and has enhanced refraction; grade 2: the mucosa is subjected to congestion and edema, is granular, has increased mucosa fragility, and is easily bleeding upon contact; grade 3: the mucosa is subjected to obvious congestion and edema, is rough, has a few spontaneous bleeding points or suffers from contact bleeding; the mucosa has relatively more inflammatory secretions, is subjected to multiple erosion and small-area ulceration; and grade 4: the mucosa is subjected to congestion and edema in a large area, is rough, suffers from obvious spontaneous bleeding and contact bleeding; and is subjected to multiple punctate erosion and large-area ulceration.

If the grade of the colonic pathology photograph of the to-be-tested drug group is low, it indicates that the drug to be tested has a good effect for treating UC.

In the present invention, the physiological basic data of the animal includes, but not limited to, a body temperature, a body weight, a stool form, an occult blood examination, a blood routine, a blood biochemical index, and a C-reactive protein.

The efficacy and therapeutic effect of the UC drug are evaluated based on changes in the physiological basic data and colon grades.

In order to make the objectives, technical solutions and advantages of the present invention more apparent, the present invention will be described in detail below with reference to embodiments.

Unless otherwise stated, the following experimental equipment and experimental reagents are used in the specific embodiments of the present invention, but they should not be construed as limiting the scope of the present invention.

### 1. Experimental Equipment

Medical image analysis system (Chongqing Ao Pu Photoelectric Technology Co., Ltd.; BL-2000);

High speed freezing centrifuge (Hunan Cenlee Scientific Instrument Co., Ltd.; TLC16M);

Ultrapure water machine (Aike Instrument; model DZG-303A); Analytical balance (Sartorius Scientific Instruments Co. , Ltd.; model BSA-124S);

Electronic endoscope (Aohua Endoscopy Co., Ltd.; VET-8015);

Automated animal hematology cell analyzer (Nanjing Perlong Pharmaceutical Co., Ltd.; XFA6030);

Macro camera: Nikon DIGITAL CAMERA model D7100, macro lens: AF-S Micro 60/2.8G ED;

No. 6 urethral catheter (HangZhou Bever Medical Devices Co., Ltd.; lot number: A16048);

An occult blood kit, injection syringes of 20 mL, 5 mL and 1 mL, a 2-mm-diameter polyethylene catheter, a vacuum blood collector, a beaker, filter paper, weighing paper, animal dissection equipment, an electronic platform scale, a zip lock bag, a garbage bag, conventional surgical instruments, and a stainless steel electronic constant-temperature water tank of model HH.W21.600.

### 2. Experimental Reagents

Glacial acetic acid (Tianjin Fuchen Chemical Reagent Co. , Ltd., lot number: 20151020);

Normal saline (Guizhou Tiandi Pharmaceutical Co., Ltd., lot number: A16021507);

Formaldehyde (Tianjin Fuchen Chemical Reagent Co. , Ltd., lot number: 20150408);

Disodium hydrogen phosphate (Tianjin Fengchuan Chemical Reagent Technology Co., Ltd., lot number: 20131008);

Sodium dihydrogen phosphate (Tianjin Fengchuan Chemical Reagent Technology Co., Ltd., lot number: 20140318);

10% MgSO₄ solution;

Weikangling capsule (Sichuan Gooddoctor Panxi Pharmaceutical Co., Ltd., lot number: 20170113);

Olsalazine capsule (Tianjing Lisheng Pharmaceutical Co., Ltd., lot number: 1608005);

Shumianning II Injection (Small Animal Disease Research Laboratory of Nanjing University, lot number: c20170225);

Starch colon-soluble capsules, and sulfasalazine colonsoluble capsules.

### Embodiment 1

I. Experimental Animal

Adult Canis lupus familiaris, aged 1 to 2 years old and weighed 6.0-12.0 kg, as provided by the Animal Hospital of Guizhou University, were fed for 2 weeks before the experiment, injected with Intervet quadruple vaccines twice (with a time interval of 2 weeks), and the healthy subjects upon clinical examination were used for this experiment.

II. Establishment of Experimental Model

1. Grouping of Experimental Dogs and Determination of Physiological Basic Data

12 healthy adult Canis lupus familiaris were randomly divided into 3 groups, 4 in each group, half male and half female. All the experimental animals were kept in a single cage during the experiment period, and fed with the same amount of food and drinking water daily, and the related data of all the Canis lupus familiaris were measured one day before modeling.

1.1 Observation of routine signs of dogs: the dogs were fed with an appropriate amount of food daily, and recorded for the body weight, body temperature, respiratory rate (beats/min), and the defection frequency, stool form, and mental state within 12 h (9:00 to 21:00).

1.2 Blood routine test and collection of normal plasma sample: after adaptive feeding, the animals were subjected to intravenous collection of anticoagulated blood in two tubes each of 4 mL and 2 mL before eating in the morning, for determining the blood biochemical indexes and C-reactive proteins of the modeling animals. take 2 times. The blood collection was conducted twice. The tubes containing whole blood was centrifuged at 3500 rmp under 4° C. for 10 min, and then the serum was removed from the tubes and stored at −80° C. for use.

2. Establishment of UC Model

The experimental animals were randomly divided into 3 groups, each being a group of 10 mL 7% acetic acid, a group of 15 mL 10% acetic acid and a group of 20 mL 5% acetic acid; on one day before modeling, the experimental animals were subjected to gavage with a 10% MgSO₄ solution at 5 mL/kg and intestinal cleansing, and were fasted but had access to water for 24 h. Subsequently, the Canis lupus familiaris were anesthetized with Shumianning II Injection at 0.1 mL/kg. The No. 6 polyethylene catheter was inserted into the colon at a depth of 20 cm from the anus of each dog, an acetic acid solution with the corresponding volume and the corresponding concentration was slowly injected into the colon, and the solution was retained in the colon for 15 s, and then the colon was rinsed with 50 mL normal saline when the dog was positioned at a head low and tail high body position. The dogs were placed back into the feeding cage at a lying down posture after the molding was completed, and the state change after the modeling was observed and recorded after the dogs naturally woke up.

3. Model Evaluation

3.1 General observation: after the modeling, the body temperature, respiratory rate (beats/min), defection frequency, stool form, and mental state of each dog were recorded daily, and the hematochezia conditions of each dog were recorded.

3.2 Colonoscopy: the dogs were respectively anesthetized with Shumianning II injection at 0.1 mL/kg on day 1, day 4, day 7, day 10 and day 14 after the modeling, and the descending colon segment and the sigmoid colon segment (at a depth of about 20 cm in the interior of the colon from the anus) of the canine colon were washed with normal saline in such a manner that the contents of the colon were washed out as much as possible. Thereafter, an electronic endoscope of model VET-8015 was inserted into the colon of the Canis lupus familiaris, and moved towards the side of anus to take photos and store the same when it reached the depth of 20 cm in the colon, so as to perform colonoscopy grading of the model.

3.3 Colon pathological section: the dogs were anesthetized with Shumianning II injection at 0.1 mL/kg on day 10 and day 14 after the modeling, and then sacrificed through air embolism. The colon was taken out, cut open, washed, and tiled, and photos thereof are taken; then the colon was cut longitudinally along the colon into three parts, the middle section of the colon was placed into a 10% formalin solution for immersion fixation, embedded in paraffin, stained with HE for preparing pathological sections, and the remaining colon was stored in a refrigerator at −80° C.

4. Modeling Results of UC Canine Model

4.1 Results of general observation: after acetic acid was given to the rectum for about 10 min, the dogs were hot and bothered, frequently looked back at the abdomen, scratched and hit the abdomen with forelimbs, and a few dogs bite the dog cages with mouth and bark due to pain, and 80% of the dogs started to have hematochezia and tenesmus at about 10-20 min, bent down and arched their backs, had frequent valsalva maneuver, frequently got around in the dog cages, had a large amount of hematochezia, where the hematochezia was mostly bleeding blood.

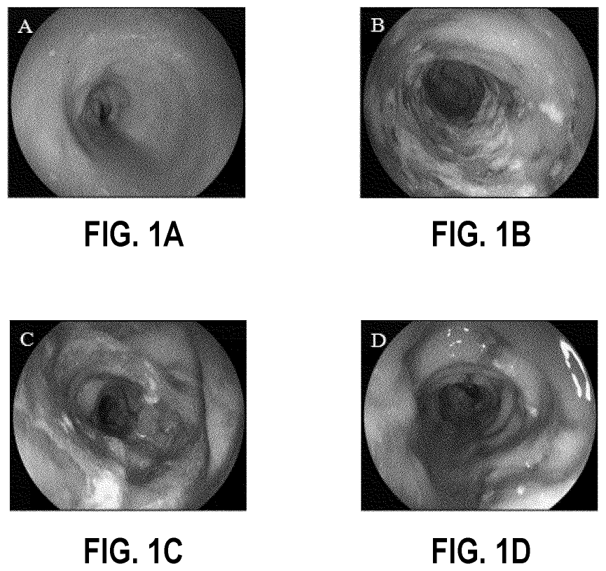

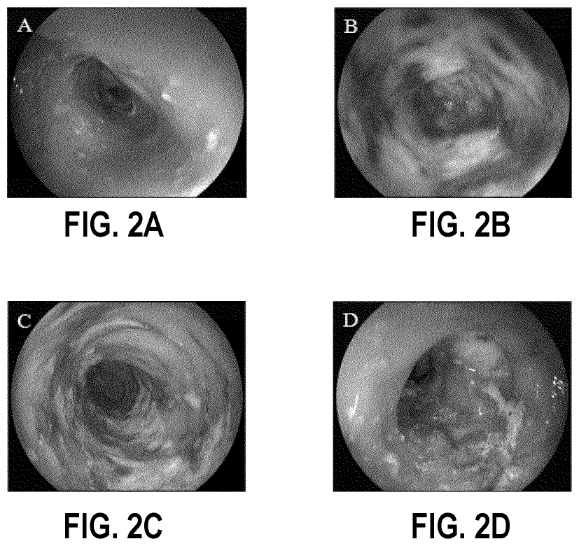

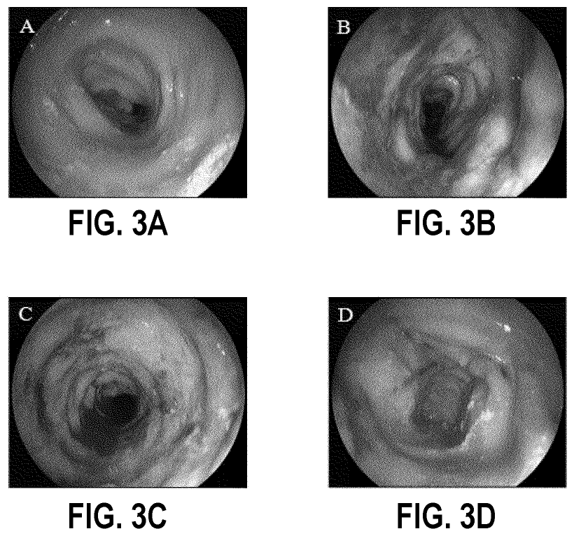

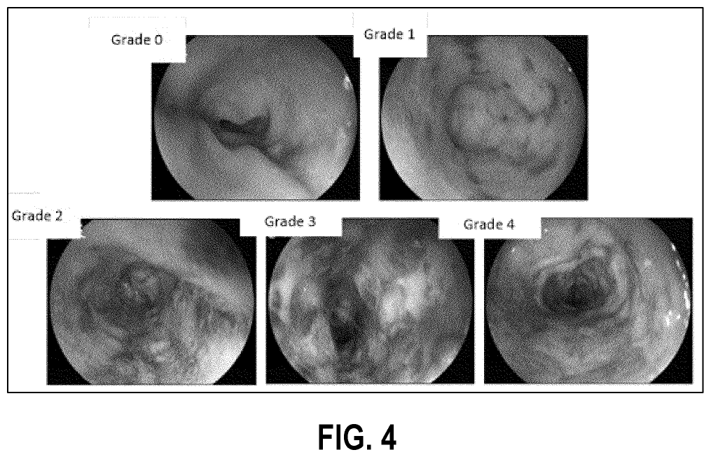

4.2 Results of colonoscopy: As shown in the endoscopy of FIGS. 1A-3D, before the modeling, the 3 groups of experimental dogs each had a smooth colonic mucosal surface which was intact and not injured, had a normal pink color, and did not show an inflammatory reaction; on day 1 after the modeling, it was visible that rectal mucosal erosion and multiple superficial ulcer, accompanied with congestion and edema, had a diffuse distribution; and on day 7 after the modeling, the endoscopy results presented that the colonic mucosa of each experimental animal was still subjected to congestion and erosion, and had a pseudomembrane as formed; the results showed that acute ulcerative inflammation occurred in the colon of each experimental animal, and thus the modeling was successful.

### Embodiment 2

Colonoscopy grading criteria of acetic acid-induced canine acute UC model

The colonic mucosa lesion during an UC active phase was subjected to diffused congestion and edema, and had blurred or disordered blood vessel textures; as the disease progressed, the mucosa became rough, had fragile fine particles diffused therein, and had natural bleeding or contact bleeding; when the lesion was worsened, multiple superficial ulcer occurred, the severe ulcer became large and deep, and mucus and blood exudation might occur in the intestinal lumen.

Currently, there are many endoscopy grading methods for evaluating UC at home and abroad. Different grading methods have different emphasis on mucosal healing and disease activity. A Baron endoscopy grading method mainly focuses on the bleeding degree of mucosa, and a Mayo colonoscopy grading method focuses on the overall characteristics of mucosa. The specific grades are as follows.

TABLE 1
Baron Endoscopic Grading

| Grades | Endoscopic Performance |
|---|---|
| 0 | The blood vessels have clearly visible textures, and has no |
| | spontaneous bleeding or contact bleeding |
| 1 | The mucosa was injured but has no bleeding, between 0-2 |
| 2 | The mucosa suffers from moderate bleeding; contact bleeding, |
| | but no spontaneous bleeding |
| 3 | Severe bleeding; spontaneous bleeding, contact bleeding |

TABLE 2
UC grade of Mayo colonoscopy

| Grades | Endoscopic Performance |
|---|---|
| 0 | The mucosa is normal or in a resting stage |
| 1 | Mild (The mucosa suffers from edema, unclear blood |
| | vessel textures, and is mildly fragile) |
| 2 | Medium (The mucosa suffers from obvious edema, |
| | disappeared blood vessel textures, and is fragile |
| | and subjected to erosion) |
| 3 | Severe (spontaneous bleeding and ulceration) |

There are no domestic criteria for animal intestinal endoscopy, and the animal spontaneous ulcerative colitis has slighter inflammation than that of the acetic acid-induced acute ulcerative colitis, such that the severity degree and disease course of the experimental animal model cannot be intuitively evaluated. Therefore, in combination with the severity degree and clinical staging of ulcerative colitis in clinical patients, focusing on mucosal injury and bleeding and redness and swollen conditions in the intestinal tract, the colonoscopy grades for acetic acid-induced acute ulcerative colitis in Canis lupus familiaris are as follows.

TABLE 3
Colonoscopy grades for acetic acid-induced acute
ulcerative colitis in Canis lupus familiaris

| Grades | Endoscopic Performance |
|---|---|
| 0 | The mucosa is pale, the vasoganglion is clear and branched. |
| | There is no redness and swollen or congestion under the mucosa, |
| | and the surface mucosa is normal. |
| 1 | The mucosa is still smooth, but is subjected to congestion and |
| | redness and swollen in a small area, and has enhanced refraction; |
| 2 | The mucosa is subjected to congestion and edema, is granular, |
| | has increased mucosa fragility, and is easily bleeding upon |
| | contact. |
| 3 | The mucosa is subjected to obvious congestion and edema, is |
| | rough, has a few spontaneous bleeding points or suffers from |
| | contact bleeding. The mucosa has relatively more inflammatory |
| | secretions, is subjected to multiple erosion and small-area |
| | ulceration. |
| 4 | The mucosa is subjected to congestion and edema in a large |
| | area, is rough, suffers from obvious spontaneous bleeding and |
| | contact bleeding. The mucosa is subjected to multiple punctate |
| | erosion and large-area ulceration. |

### Embodiment 3

1. Selection of Experimental Dogs

10 healthy purebred Golden Retriever dogs, 10 healthy purebred Beagle dogs, and 10 Canis lupus familiaris dogs were selected, where the dogs were male and female in each half, and were 1 to 2 years old; during the experiment, the three kinds of experimental dogs were respectively fed in three separate and identical experiment environments and housed in individual cages.

2. Model Establishment of Acetic Acid-Induced Ulcerative Colitis in Experimental Dogs

On one day before modeling, the experimental dogs were subjected to gavage with a 10% MgSO₄ solution at 5 mL/kg and intestinal cleansing, and were fasted but had access to water for 24 h. Subsequently, the experimental dogs were anesthetized by intramuscularly injecting Shumianning at 0.15 mL/kg. A polyethylene catheter was inserted into the colon at a depth of 20 cm from the anus of each dog, 20 mL of a 7% acetic acid solution was slowly injected into the colon, and the solution was retained in the colon for 1 min, and then the colon was rinsed with 50 mL normal saline when the dog was positioned at a head low and tail high body position.

3. Observation of Complications and Survival Rates in Experimental Dogs

3.1 Detection of complications: respectively on day 2, day 4, day 6, day 8 and day 10 after the modeling, the feces of the experimental dogs were taken and detected for infection conditions of canine distemper virus and canine parvovirus; and the body weights and general health conditions of the experimental dogs were recorded.

3.2 Survival Records: the survival conditions of the experimental dogs were observed daily after the modeling.

4. Results

4.1 Detection of complications: for the purebred Golden Retriever dogs, on day 4, 2 dogs were detected as positive in canine distemper virus, and 2 dogs were detected as positive in canine parvovirus; on day 8, 3 dogs were detected as positive in canine distemper virus, and 6 dogs were detected as positive in canine parvovirus; and on day 10, 5 dogs were detected as positive in canine distemper virus, and 9 dogs were detected as positive in canine parvovirus. For the purebred Beagle dogs, on day 6, 2 dogs were detected as positive in canine distemper virus, and 3 dogs were detected as positive in canine parvovirus; and on day 10, 3 dogs were detected as positive in canine distemper virus, and 5 dogs were detected as positive in canine parvovirus. For the purebred Canis lupus familiaris dogs, on day 2 to day 10 after the modeling, no dog was detected as positive in canine distemper virus or canine parvovirus.

4.2 Survival rate: On day 6 after the modeling, death occurs in the purebred Golden Retriever dogs, where before death the dogs were foaming at the mouth and had a large amount of excrement at the anus; On day 8 after the modeling, death occurs in both the purebred Golden Retriever dogs and the purebred Beagle dogs, where before death the dogs were foaming at the mouth and had a large amount of reddish brown excrement at the anus. On day 10 after the modeling, 4 purebred Golden Retriever dogs survived with a survival rate of 40%, where the survived Golden Retriever dogs had a reduced diet capacity, were of weak and feeble states, had pus-like gum at the canthus, were wheezed, and could be observed with a multiple-mucous-stool defecation phenomenon; 8 purebred Beagle dogs survived with a survival rate of 80%, where the survived Beagle dogs had a normal diet capacity, but most of them were weak and seldom moved, some of the dogs had pus-like gum at the canthus, breathed normally, and could be observed with a multiple-mucous-stool defecation phenomenon; and 10 purebred Canis lupus familiaris dogs survived with a survival rate of 100%, where the survived dogs had normal diet and activities, normal canthus, breathed normally, and could be observed with a multiple-loose-stool or multiple-mucous-stool defecation phenomenon.

5. Conclusion

It can be determined from the defecation and other related symptoms of the experimental dogs that still survived on day 10 after the modeling that, the UC modeling method used in this experiment can successfully induce ulcerative colitis in the 3 kinds of dogs. That is, this experiment verified the feasibility of using the UC canine model for evaluating the efficacy of a drug.

The canine parvovirus infection is also referred to as canine parvoviral enteritis or canine hemorrhagic colitis, is divided into two types, i.e., an enteric type and a myocardial type, and is characterized by acute hemorrhagic enteritis and non-purulent myocarditis in clinical manifestations. The enteric type occurs in adult dogs and puppies older than 3 months. The virus invades the intestinal membrane, causing loss of appetite, emesis, bloody stools and rise of body temperature in the canine patients. The myocardial type usually occurs in puppies, where the virus invades the cardiac muscles and usually causes a death within 72 hours. The canine distemper virus infection has a variety of manifested symptoms associated with viral virulence, environmental conditions, age and immune status of the host, and can be divided into 5 characteristic types, i.e., a hyperacute type, an acute type, a type having gastrointestinal symptoms, a type having neurological symptoms and a type having skin symptoms, where once the characteristic symptoms appear, the prognosis is extremely poor.

In this experiment the experimental animals were fed in single cages, the rearing environment was disinfected every day, and the feeders were specially trained, so that in this experiment the infection with the canine parvovirus and the canine distemper fever virus in the Golden Retriever dogs and the Beagle dogs is mainly caused by the reason that the 2 types of dogs are more susceptible to these types of viruses than the Canis lupus familiaris dogs.

The main symptom of adult dogs infected with the canine parvovirus and the canine distemper fever virus is acute enteritis, and thus complications of the 2 kinds of virus infection will interfere with efficacy evaluation to a certain extent; and additionally, the high mortality of the 2 kinds of virus infection complications causes increased cost of the animal experiment.

Based on the acetic acid-induced UC model, for the Golden Retriever dogs and the Beagle dogs, there are a higher proportion of complications and death. Therefore, this model uses the Canis lupus familiaris dog, which has a stronger ability of anti-infection, a smaller body size and thus easier to model, as the experiment animal.

### Embodiment 4

I. Experimental Model Verification

1. Grouping of Experimental Dogs and Determination of Physiological Basic Data

16 qualified adult Canis lupus familiaris were randomly divided into 4 groups, 4 in each group, half male and half female. All the experimental animals were kept in a single cage during the experiment period, and fed with the same amount of food and drinking water daily, and the related data of all the Canis lupus familiaris were measured one day before modeling.

2. Model Establishment of Acetic Acid-Induced Ulcerative Colitis in Canis lupus Familiaris Dogs

On one day before modeling, the experimental dogs were subjected to gavage with a 10% MgSO₄ solution at 5 mL/kg and intestinal cleansing, and were fasted but had access to water for 24 h. Subsequently, the experimental dogs were anesthetized by intramuscularly injecting Shumianning at 0.15 mL/kg. A polyethylene catheter was inserted into the colon at a depth of 20 cm from the anus of each dog, 20 mL of a 7% acetic acid solution was slowly injected into the colon, and the solution was retained in the colon for 1 min, and then the colon was rinsed with 50 mL normal saline when the dog was positioned at a head low and tail high body position.

3. Mode of Administration

From day 1 after the modeling, the drug was administrated to the dogs at a dose of 2 capsules/kg body weight in the morning and evening through fasting gavage, the dogs had access to food intake 2 h after the administration, and the administration was conducted successively for 7 d; where group A (the modeled group) was fed with the starch colon-soluble capsules, group B was fed with the weikangling capsules, group C was fed with the olsalazine capsules, and group D was fed with sulfasalazine colonsoluble capsules.

4. Determination of Efficacy

4.1 General Observation: Electronic endoscopy was used for observation on day 1 before and after the modeling, and on day 3 and day 7 after administration, where whether ulceration, fester, bleeding and the like occur in the colonic mucosa was recorded; each group was graded with a Disease Activity Index (DAI); and the body temperature, body weight and stool form of each experimental dog was recorded. The DAI grading method is as shown in Table 4.

TABLE 4
Grading of Disease Activity Index (DAI)

| | Loss of | | Occult blood |
|---|---|---|---|
| Grades | weight (%) | Stool form | conditions |
| 0 | <1 | normal | occult |
| | | | blood (−) |
| 1 | 1-5 | between normal | occult |
| | | and mushy stool | blood (+) |
| 2 | 5.01-10 | mushy stool | occult |
| | | | blood (++) |
| 3 | 10.01-15 | between mushy | occult |
| | | stool and | blood (+++) |
| | | diarrhea | |
| 4 | >15 | diarrhea | bleeding visible |
| | | | to the naked |
| | | | eyes |

Note:
Normal feces = shapeable feces; mushy stool = mushy feces that do not stick to the anus; and diarrhea = liquefied feces sticking to the anus.
Loss of body weight (%) = (the body weight at a certain time point − the body weight before the modeling)/the body weight before the modeling × 100%
DAI = (a grade for loss of body weight + a grade for stool form + a grade for occult blood condition)/3.

4.2 Occult Blood Examination: Stool occult blood (also known as fecal occult blood) refers to the condition in which there is a small amount of bleeding in the digestive tract, red blood cells are destroyed by digestion, the feces have no abnormal changes in the appearance, and the bleeding cannot be confirmed either by the naked eye or under the microscope. An occult blood kit was used for detection, where a small amount of stool was picked up with a cotton swab and placed onto a white porcelain board, added dropwise with an o-tolidine solution, and then dropwise with a hydrogen peroxide solution, and the occult blood grading was conducted according to the developing time and the developing degree.

4.3 Blood Routine: A blood cell analyzer was used to analyze the blood of the experimental animal for the number of total white blood cells (WBC), the number of total lymphocytes (LYM), the number of mononuclear cells (MONO), the number of total neutrophile granulocytes (GRAN), the percentage of lymphocytes (LYM %), the percentage of mononuclear cells (MON %), the percentage of neutrophile granulocytes (GRA %), hemoglobin (HGB), hematocrit (HCT), the number of total red blood cells (RBC), a mean corpuscular volume (MCV), a mean corpuscular-hemoglobin concentration (MCH), a mean corpuscular hemoglobin concentration (MCHC), a red blood cell distribution width (RDW %), the count of platelet (PLT), and a mean platelet volume (MPV) in the blood of the experimental animal.

4.4 Blood Biochemical Index: An IDEXX biochemical analyzer was used to analyze the serum for contents of cholesterol (CHOL), total protein (TP), albumin (ALB), blood urea nitrogen (BUN), creatinine (CREA), glucose (GLU), total bilirubin (TBIL), blood calcium (CA), phosphate radicals (PHOS), Alanine transaminase (ALT), alkaline phosphatase (ALKP), amylase (AMYL), and globulin (GLOB).

4.5 C-reactive Protein (CRP): An USHIO INC Point-reader V was used to detect the serum for the content of C-reactive protein in the serum before the modeling, and on day 1, day 4 and day 7 after the modeling.

4.6 Observation of pathological sections under optical microscope (HE): Sections were prepared for observation of colonic mucous epithelium: the epithelial cells had normal morphology; and infiltration and the like conditions occur in goblet cells, crypt cells, and inflammatory cells.

5. Results of Treating the Inflammatory Canine UC Model with a Drug Used for Cinically Treating UC

5.1 Results of General Observation: After acetic acid was given to the rectum, at about 10 min, the dogs were hot and bothered, frequently looked back at the abdomen, scratched and hit the abdomen with forelimbs, and a few dogs bite the dog cages with mouth and bark due to pain, and at about 10-20 min, 80% of the dogs started to have hematochezia and tenesmus, bent down and arched their backs, had frequent valsalva maneuver, frequently got around in the dog cages, had a large amount of hematochezia, where the hematochezia was mostly bleeding blood.

5.2 Endoscopy: Before the modeling, the colonic mucosa was presented pink and the mucosal surface was smooth. On day 1 after the modeling, it could be seen that rectal mucosal erosion and multiple superficial ulcer, accompanied with congestion and edema, had a diffuse distribution in the modeled group.

For the modeled group, on day 4 after the modeling, in group A (the modeled group), the colonic mucosa was subjected to congestion and edema, the mucosal surface became rough, fine particles with relatively constant size and diffuse distribution appear, the tissue became fragile, there was natural bleeding or contact bleeding, and there was mucinous secretions in the colonic lumen; and on day 7 after the modeling, in the modeled group, the colonic mucosa was still subjected to congestion and erosion, and a pseudomembrane was formed.

Endoscopy results of the drug groups: on day 4 after administration, in group B (the weikangling capsule group), the colonic mucosa was subjected to erosion and edema, and there were still ulcers; in group C (the olsalazine capsule group), the colonic mucosa was subjected to reduced edema, and the ulcers were smaller and less; and in group D (the sulfasalazine colonsoluble capsule group), it was visible that many small red spots diffused in the colonic mucosa and presented in needle-like or patch-like forms, had irregular morphology, and were arranged irregularly.

On day 7 after administration, in group B, the colonic mucosa was subjected to slight edema, and the ulcers were healed; in group C, the exudates were absorbed, the color of the intestinal mucosa became pale, the mucosa had a normal luster but was not fully recovered; and in group D, it was visible that the colon contained pus, but the colonic mucosal surface was smooth, and no ulcerative lesion was seen.

5.3 DAI grading results

TABLE 5
DAI grading results

| | Grading time | | | |
|---|---|---|---|---|
| | 1 d before | 1 d after | 4 d after | 7 d after |
| Groups | modeling | modeling | administration | administration |
| Group A | 0 | 9.08 | 7.17 | 5.67 |
| Group B | 0 | 8.94 | 4.17 | 1.64 |
| Group C | 0 | 9.00 | 5.84 | 2.33 |
| Group D | 0 | 8.92 | 5.33 | 3.67 |

Note: Each data in the table is the average value of DAIs of experimental animals in each group.

DAI grading results: as can be seen from Table 5, the DAI value was the highest on day 1 after the modeling, and the DAI was gradually decreased from day 4 to day 7 after administration. The modeled group has the highest DAI value compared with other groups in the corresponding period.

5.4 Fecal occult blood results: Results of occult blood experiment: on one day before the modeling, the canine stool was detected as negative (−) in the occult blood detection; on day 1 and day 4 after administration, the canine stool was detected as strong positive (+++) in the occult blood experiment, and bleeding-blood-stained stool was visible by the naked eyes; and on day 7 after administration, the canine stool was still detected as positive (++) in the occult blood detection, but the bleeding condition was alleviated.

5.5 Results of blood routine examination: The result of white blood cell changes was shown in Table 6. On day 4 after administration, the number of total white blood cells (WBC) in each of group A (the modeled group) and group C was higher, while the WBC in each of group B and group D was recovered into the normal range; and on day 7 after administration, the WBC in each of group B and group D was maintained in the normal range, while the WBC in group C was continually decreased to near the normal range.

TABLE 6
WBC detection results (mean ± standard deviation, n = 4)

| | Collection Time | | |
|---|---|---|---|
| | | | (×10⁹/L) |
| | 1 d after | | 7 d after |
| | modeling | 4 d after | administration |
| Groups | (×10⁹/L) | administration | (×10⁹/L) |
| Group A | 23.825 ± 3.113 | 24.275 ± 4.182 | 21.075 ± 3.004 |
| Group B | 23.805 ± 13.831 | 16.800 ± 4.645 | 13.550 ± 2.767 |
| Group C | 24.275 ± 4.920 | 21.823 ± 7.889 | 18.050 ± 0.893 |
| Group D | 25.000 ± 12.884 | 11.250 ± 5.540 | 10.600 ± 2.688 |

Note:
the reference range of WBC was 6 × 10⁹/L-17 × 10⁹/L.

5.6 Results of Biochemical Detection: Among biochemical detection indexes, only BUN changed, and during the experiment BUN was within the normal range or slightly lower than the normal range. The remaining biochemical detection indexes did not differ at each detection time point.

5.7 Detection results of C-reactive protein (CRP): The result of CRP changes was shown in Table 7. On day 4 after administration, the CRP was decreased in each group treated with a drug, where the decrease in group D was the most significant; and on day 7 after administration, the CRP of group D was 7.975±2.425, which was recovered into the normal range.

TABLE 7
CRP Detection Results (mg/L) (mean ± standard deviation, n = 4)

| | Collection Time | | |
|---|---|---|---|
| | 1 d after | 4 d after | 7 d after |
| Groups | modeling | administration | administration |
| Group A | 103.775 ± 16.747 | 97.600 ± 29.676 | 36.425 ± 8.764 |
| Group B | 99.125 ± 23.367 | 67.450 ± 41.758 | 14.500 ± 2.783 |
| Group C | 93.575 ± 17.544 | 60.125 ± 18.164 | 18.450 ± 7.303 |
| Group D | 103.675 ± 28.430 | 48.150 ± 19.549 | 7.975 ± 2.425 |

Note:
the reference range of CRP was 1-10 mg/L.

In embodiments of the present invention, a canine UC model was made by perfusing 10-20 mL of a 5-10% acetic acid solution into the rectum, and the endoscopy results show that acute UC was successfully induced in the experimental animal. The results of verifying the use of the model in efficacy evaluation by using clinical first-line drugs weikangling, olsalazine and sulfasalazine showed that: on day 4 after administration, in the weikangling capsule group, the colonic mucosa was subjected to erosion and edema, and there were still ulcers; in the olsalazine capsule group, the colonic mucosa was subjected to reduced edema, and the ulcers were smaller and less; and in the sulfasalazine colonsoluble capsule group, it was visible that many small red spots diffused in the colonic mucosa and presented in needle-like or patch-like forms, had irregular morphology, and were arranged irregularly; and on day 7 after administration, in the weikangling capsule group, the colonic mucosa was subjected to slight edema, and the ulcers were healed; in the olsalazine capsule group, the exudates were absorbed, the color of the intestinal mucosa became pale, the mucosa had a normal luster but was not fully recovered; and in the sulfasalazine colonsoluble capsule group, it was visible that the colon contained pus, but the colonic mucosal surface was smooth, and no ulcerative lesion was seen. 4 dogs in the modeled group had loose and blood-stained stool, and showed obvious inflammatory symptoms in colonic morphology and histological observations. At the same time, it was found through histological observation on day 3 and day 7 after the modeling that, on day 3 after the modeling the ulcer lesions were still obvious, and there was a significant inflammatory cell infiltration; and on day 7 after the modeling it was visible that the ulcer lesions were decreased or even fallen off, the inflammatory cell infiltration was significantly reduced, and a mass fibroblast proliferation occurred, which indicated that the experimental animal was undergoing a self-healing process. For the experimental dogs treated with clinical UC-treating drugs in the corresponding time period, on day 3 after administration the ulcers were decreased much more as compared with those of the modeled group, and the inflammatory cell infiltration was also greatly reduced, which indicated that the inflammations of the colon were improved and the colonic mucosa was gradually recovered.

The embodiments described above are only descriptions of preferred embodiments of the present invention and are not intended to limit the scope of the present invention. Various variations and modifications can be made to the technical solution of the present invention by those of ordinary skill in the art, without departing from the design and spirit of the present invention. The variations and modifications should all fall within the claimed scope defined by the claims of the present invention.

1. A method for establishing an ulcerative colitis animal model, comprising:
making a Canis lupus familiaris be subjected to gavage and intestinal cleansing, and be fasted but have access to water for 24 h; and
anesthetizing the Canis lupus familiaris, and then making the anesthetized Canis lupus familiaris be subjected to enema with an acetic acid solution, to obtain the ulcerative colitis animal model when the Canis lupus familiaris naturally wakes up.

2. The method of claim 1, wherein the Canis lupus familiaris is an adult dog.

3. The method of claim 2, wherein the Canis lupus familiaris is 1-2 years old.

4. The method of claim 2, wherein a body weight of the Canis lupus familiaris is 6.0-12.0 Kg.

5. The method of claim 1, wherein an enema manner is injecting the acetic acid solution into a colon of the Canis lupus familiaris, retaining the acetic acid solution in the colon for more than 10s, and then rinsing the colon with normal saline.

6. The method of claim 5, wherein a mass concentration of the acetic acid solution is 5-10%.

7. The method of claim 6, wherein a dose of the acetic acid solution is 1 mL to 2 mL per kilogram of body weight.

8. The method of claim 1, wherein a gavage drug is an MgSO4 solution.

9. The method of claim 8, wherein the MgSO4 solution has a concentration of 5-10%, and a dose of 5-10 mL/kg.

10. The method of claim 1, wherein the anesthetizing the Canis lupus familiaris comprises: intramuscularly injecting Shumianning into the Canis lupus familiaris.

11. A use of the ulcerative colitis animal model established by the method of claim 1 in screening of UC therapeutic drugs.

12. A method for screening and evaluating UC therapeutic drugs, comprising the:
setting a to-be-tested drug group, a normal saline control group, and a normal control group, wherein the to-be-tested drug group and the normal saline control group use the ulcerative colitis animal model established by using the method of claim 1, and the normal control group contains a healthy Canis lupus familiaris not subjected to the modeling; observing and recording physiological basic data and colonic pathology conditions of the to-be-tested drug group, the normal saline control group and the normal control group;
feeding the to-be-tested drug group with a drug to be tested, and feeding both the normal saline control group and the normal control group with normal saline, and observing and recording the physiological basic data and colonic pathology conditions of the to-be-tested drug group, the normal saline control group and the normal control group after a treatment; and
analyzing the data and grades of colonic pathological photographs recorded in the setting and feeding steps, and evaluating a therapeutic effect of the drug to be tested; wherein if the physiological basic data of the to-be-tested drug group is closer to the corresponding measurement value of the normal control group than the physiological basic data of the normal saline control group, or the grade of the colonic pathological photograph of the to-be-tested drug group is lower than that of the normal saline control group, then it indicates that the drug to be tested has a therapeutic effect of treating UC.

13. The method of claim 12, wherein a grading criteria of the colonic pathology photographs are:
grade 0: a mucosa is pale, a vasoganglion is clear and branched; there is no redness and swollen or congestion under the mucosa, and the surface mucosa is normal;
grade 1: the mucosa is still smooth, but is subjected to congestion and redness and swollen in a small area, and has enhanced refraction;
grade 2: the mucosa is subjected to congestion and edema, is granular, has increased mucosa fragility, and is easily bleeding upon contact;
grade 3: the mucosa is subjected to obvious congestion and edema, is rough, has a few spontaneous bleeding points or suffers from contact bleeding; the mucosa has relatively more inflammatory secretions, is subjected to multiple erosion and small-area ulceration; and
grade 4: the mucosa is subjected to congestion and edema in a large area, is rough, suffers from obvious spontaneous bleeding and contact bleeding; and is subjected to multiple punctate erosion and large-area ulceration;
wherein if the grade of the colonic pathology photograph of the to-be-tested drug group is low, it indicates that the drug to be tested has a good effect for treating UC.

14. The method of claim 12, wherein the physiological basic data comprises one or more of a body temperature, a body weight, a stool form, an occult blood examination, a blood routine, a blood biochemical index, and a C-reactive protein.

15. The method of claim 3, wherein a body weight of the Canis lupus familiaris is 6.0-12.0 Kg.

16. The use of the ulcerative colitis animal model of claim 11 in screening of UC therapeutic drugs, wherein the Canis lupus familiaris is an adult dog.

17. The use of the ulcerative colitis animal model of claim 16 in screening of UC therapeutic drugs, wherein the Canis lupus familiaris is 1-2 years old.

18. The use of the ulcerative colitis animal model of claim 17 in screening of UC therapeutic drugs, wherein a body weight of the Canis lupus familiaris is 6.0-12.0 Kg.

19. The use of the ulcerative colitis animal model of claim 16 in screening of UC therapeutic drugs, wherein a body weight of the Canis lupus familiaris is 6.0-12.0 Kg.

20. The use of the ulcerative colitis animal model of claim 11 in screening of UC therapeutic drugs, wherein an enema manner is injecting the acetic acid solution into a colon of the Canis lupus familiaris, retaining the acetic acid solution in the colon for more than 10s, and then rinsing the colon with normal saline.
